(19) United States
(12) Patent Application Publication
(10) Pub. No.: US 2017/0210641 A1
(43) Pub. Date: Jul. 27, 2017

(54) BIOSORBENTS FOR REMOVAL OF CONTAMINANTS FROM WASTE/OIL SAND PROCESS-AFFECTED WATER (OSPW) AND CONSOLIDATION OF OIL SANDS TAILINGS

(71) Applicant: THE GOVERNORS OF THE UNIVERSITY OF ALBERTA, Edmonton (CA)

(72) Inventors: Aman ULLAH, Edmonton (CA); Tariq SIDDIQUE, Edmonton (CA)

(73) Assignee: THE GOVERNORS OF THE UNIVERSITY OF ALBERTA, Edmonton (CA)

(21) Appl. No.: 15/379,291

(22) Filed: Dec. 14, 2016

Related U.S. Application Data
(60) Provisional application No. 62/266,772, filed on Dec. 14, 2015.

(51) Int. Cl. C02F 1/28; B01J 20/24; C07K 14/47; C02F 1/52

(52) U.S. Cl. CPC C02F 1/286; C02F 1/5272; B01J 20/24; C07K 14/4741; C02F 2101/20

(57) ABSTRACT

Methods of using modified keratin to remove trace metals and/or napthenic acids from a waste water stream are described. The waste water stream may be mature or fine fluid tailings from oil sands operations, and the methods may include the removal of trace metals, naphthenic acids with simultaneous consolidation of the tailings. The keratin may be modified to unfold the keratin, disrupt disulphide bonding, add functional groups, and/or maintain or stabilize the unfolded configuration.

### FIELD OF THE INVENTION

This invention relates to a biosorbent comprising modified or unmodified keratin, and its use to treat waste water such as oil sands processed water (OSPW) and consolidate oil sands tailings.

### BACKGROUND

Large crude oil reserves are found in oil sands deposits. Water is used in the extraction of crude oil from oil sands and the use of freshwater in surface mining operations is increasing. Water recovered from hot water extraction process is called oil sands processed water (OSPW). OSPW is acutely toxic to aquatic biota, and oil processing companies cannot discharge OSPW to other water receiving bodies. One pressing challenge in the oil sand sector is to limit the use of fresh water and maintain good water quality.

Due to increasing environmental concerns and legal regulations, oil sand industries are using water in repeated extraction cycles in order to improve water use efficiency, which leads to a decline in water quality and reduces extraction efficiency due to contamination. In addition to metals, the main toxic component in OSPW are the naphthenic acids which are in the acidic fraction of dissolved organic matter.

As well, the treatment and disposal of millions of cubic meters of toxic processed water and tailings currently stored in large tailings ponds and consolidation of tailings ponds to transform them into reclaimed land is another challenge for oil sand industries. Therefore, recovering water from tailings for re-use and consolidating the tailings solids to decrease the volume of stored tailings are two major challenges which plague the oil sands surface mining industry. Chemical additives (e.g., gypsum) used to consolidate the ‘fines’ can deteriorate the quality of recovered water for re-cycling and can cause unanticipated hazardous side-effects in the ponds (e.g., biogenic hydrogen sulphide), while physical treatments such as centrifugation are cost- and energy-intensive.

To address these challenges, research has focused on processed water remediation and consolidation technologies. Several treatment technologies have been developed over the years, however, few techniques have demonstrated significant improvements with respect to the treatment of OSPW. Known techniques include adsorption, biological treatment, advanced oxidation, membrane processes, and wetland treatment. Among these methods, adsorption methods are considered an effective processing method for the removal of both heavy metal ions and other major contaminants. The flexibility, high removing ability and recyclability for the adsorbent materials makes adsorption a widely applied treatment for water remediation. The most common adsorbents which have been used to treat oilfield produced water include activated carbon, natural organic matter, and synthetic polymers. Activated carbon has been effective in adsorption of naphthenic acids to a certain level under specific pH conditions, but poor removal rates have been observed for other target pollutants. Synthetic polymers such as polyethylene terephthalate (PET), polystyrenes, polyacrylics such as polyacrylamides have been used as adsorbents. These polymers are more effective in removal of organics compared to heavy metals, however, they are completely non-degradable and not considered to be environmentally friendly. Further, the addition of flocculants and coagulants such as polyacrylamide to alter tailings properties (e.g., www.suncor.com/tailings) has unknown long-term stability and environmental impact (e.g., polymer degradation to toxic acrylamide)

Use of natural polymers for the adsorption of contaminants can be a viable option. Some biopolymers such as chitin and chitosan have already been reported as efficient heavy metal scavengers due to the presence of hydroxyl and amino functional groups.

Wool keratin has been used in some studies for removal of heavy metals such as chromium, and aluminum from synthetically contaminated water. Chicken feathers may remove heavy metal ions and organic dyes from contaminated/wastewaters due to their high surface area and several reactive functional groups. Keratin fiber has been used to adsorb heavy metals (copper, lead, chromium, mercury and uranium) from synthetic dilute solutions of these metals, and it has been reported that keratin fiber has a good capacity as a medium for removal of heavy metals from water. However, there are limitations in the uptake of metals, which were mainly attributed to difficulty in unfolding and mixing keratin in aqueous solution because of its hydrophobic and crosslinked structure. In addition, keratin or modified keratin has not been reported to enhance the flocculation/consolidation of tailings.

It is commonly believed that electrostatic interactions, metal chelation and ion pairs formation are the main mechanisms believed to occur when a metal is adsorbed by a biopolymer such as keratin. Surface adsorption or adsorption complexation may also occur due to ion-exchange, hydrogen bonds, hydrophobic and van der Waals interactions. The side chains of amino acids do not participate in polypeptide formation and therefore are free to interact with their environment.

There remains a need in the art to identify and implement cost-effective methods and materials to remove contaminants from OSPW, other industrial waste waters and particularly simultaneous removal of contaminants from and consolidation of oil sands tailings.

### SUMMARY OF THE INVENTION

In one aspect, the invention comprises keratin based absorbents, and methods of making them.

In another aspect, the invention comprises a method of removing trace metals and/or napthenic acids from a waste water stream, comprising the step of contacting the waste water stream with keratin or modified keratin.

In one embodiment, the method involves simultaneously removing contaminants from and consolidating oil sands tailings.

In one embodiment, the keratin is modified by a denaturing step, such as by unfolding and breaking of disulfide linkages, and precipitated at about its isoelectric point.

In one embodiment, the keratin is further modified by:
a polyol such as glycerol ethoxylate, or
a thiol-bearing cage structure, such as a silsesquioxane, such as polyhedral oligomeric silsesquioxane (POSS) cage with organic hydrophobic substituents and/or hydroxyl, carboxyl or amine substituents, or
aliphatic or aromatic amines, such as dimethylamino-1-propylamine, or
iron based minerals, clays or layered silicates which bear multiple hydroxyl groups, such as smectites, vermiculite, kaolins, illite, chlorite, or iron oxyhydroxides such as hematite, goethite, lepidcrocite or ferrihydrite, or
an organic polyacid, such as tannic acid or citric acid.

### BRIEF DESCRIPTION OF THE DRAWINGS

In the drawings, like elements are assigned like reference numerals. The drawings are not necessarily to scale, with the emphasis instead placed upon the principles of the present invention. Additionally, each of the embodiments depicted are but one of a number of possible arrangements utilizing the fundamental concepts of the present invention. The drawings are briefly described as follows:

FIGS. 1A-1C show scanning electron microscope (SEM) photos of pure keratin fiber (1A), SM-01 (1B) and SM-06 (1C),

FIG. 2 shows WAXD patterns of neat processed feather (SM-03) compared with solution modified (SM 01, 02, 04, 05, 06) materials;

FIG. 3 shows FTIR spectra of neat processed feather compared with solution modified (SM 01, 02, 04, 05, 06) materials;

FIG. 4 shows DSC heat flow signals of neat processed feather compared with solution modified (SM 01, 02, 04, 05, 06) materials;

FIG. 5A shows TGA curves of neat processed feather (SM-03) compared with solution modified (SM 01, 02, 04, 05, 06) materials (FIG. 5B);

FIG. 6 shows Biosorbent SM-01; “Before” concentrations indicate the amount of a certain element before contact with biosorbent. “After” concentration is the amount left in solution after 24 hours in contact with the sorbent. Control is for the amount of the element that leaches from sorbent during shaking with millipore water;

FIG. 7 shows Biosorbent SM-03; “Before” concentrations indicate the amount of a certain element before contact with biosorbent. “After” concentration is the amount left in solution after 24 hours in contact with the sorbent. Control is for the amount of the element that leaches from sorbent during shaking with millipore water;

FIG. 8 shows Biosorbent SM-06; “Before” concentrations indicate the amount of a certain element before contact with biosorbent. “After” concentration is the amount left in solution after 24 hours in contact with the sorbent. Control is for the amount of the element that leaches from sorbent during shaking with millipore water;

FIGS. 9A-9C show sorptions of Ca and Na concentrations by SM-01 (9A), SM-03 (9B), and SM-06 (9C)—“Before” concentrations indicate the amount of a certain element before contact with biosorbent. “After” concentration is the amount left in solution after 24 hours in contact with the sorbent. Control is for the amount of the element that leaches from sorbent during shaking with millipore water;

FIGS. 10A-10C show treatment of OSPW with sample SM-01 (10A), SM-03 (10B) and SM-06 (10C). Graph I displays the treatment of oil sands process-affected water without any addition of metals V, Cr, Ni or Se. Graph II displays the sorption of these elements when the sample is spiked with high concentration of metals;

FIGS. 11A-11B show FTIR spectra of naphthenic acids adsorption by different sorbents. The monomers of the naphthenic acid carboxylic group absorb at 1743 cm-1 and the hydrogen-bonded dimers absorb at 1705 cm-1 (11A) and calibration plot of naphthenic acids (NAs) standards (11B);

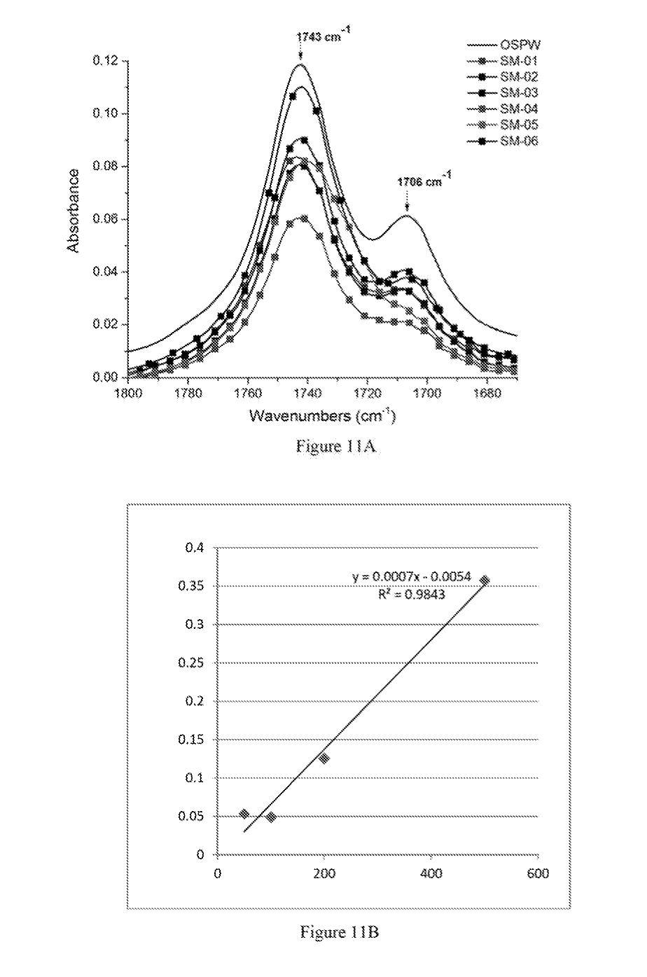

FIG. 12 shows relative settling of fluid fine tailings (FFT) with different flocculants.

FIG. 13 shows graphs of water recovery and consolidation rates of FFT using different flocculants.

### DETAILED DESCRIPTION OF PREFERRED EMBODIMENTS

Keratin is an animal fibrous protein and is present in feathers of birds such as chickens and turkeys. Feathers contain more than 90% keratin protein. Currently, the majority of the feathers are disposed in landfills or used as animal feed. Embodiments of the invention are based on observations of structural changes during modification of poultry feathers and the effect of modification on sorption of various trace metals and naphthenic acids from OSPW and contaminated water. Chemically, several modifications of keratin were achieved by treating with different modifying agents.

Without restriction to a theory, keratin has several amino acid side chains which are believed to interact with metals and adsorb them, and also has hydrophobic chains which are believed to interact with other contaminants such as naphthenic acids and adsorb them.

References in the specification to “one embodiment”, “an embodiment”, etc., indicate that the embodiment described may include a particular aspect, feature, structure, or characteristic, but not every embodiment necessarily includes that aspect, feature, structure, or characteristic. Moreover, such phrases may, but do not necessarily, refer to the same embodiment referred to in other portions of the specification. Further, when a particular aspect, feature, structure, or characteristic is described in connection with an embodiment, it is within the knowledge of one skilled in the art to affect or connect such aspect, feature, structure, or characteristic with other embodiments, whether or not explicitly described.

The singular forms “a,” “an,” and “the” include plural reference unless the context clearly dictates otherwise. It is further noted that the claims may be drafted to exclude any optional element. As such, this statement is intended to serve as antecedent basis for the use of exclusive terminology, such as “solely,” “only,” and the like, in connection with the recitation of claim elements or use of a “negative” limitation. The term “and/or” means any one of the items, any combination of the items, or all of the items with which this term is associated.

The term “about” may refer to a variation of up to ± 10% of the value specified. For example, “about 50 percent” can in some embodiments carry a variation from 45 to 55 percent. For integer ranges, the term “about” can include one or two integers greater than and/or less than a recited integer at each end of the range. Unless indicated otherwise herein, the term “about” is intended to include values and ranges proximate to the recited value or range that are equivalent in terms of the functionality of the composition, or the embodiment. As will be understood by the skilled artisan, all numbers, including those expressing quantities of reagents or ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percents or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc.

As will also be understood by one skilled in the art, all language such as “up to”, “at least”, “greater than”, “less than”, “more than”, “or more”, and the like, include the value recited and such terms refer to ranges that can be subsequently broken down into sub-ranges as discussed above. In the same manner, all ratios recited herein also include all sub-ratios failing within the broader ratio. Accordingly, specific values recited for radicals, substituents, and ranges, are for illustration only; they do not exclude other defined values or other values within defined ranges for radicals and substituents.

In one aspect, the invention comprises the use of keratin, such as avian keratin, modified or unmodified, to selective adsorb trace metals and/or napthenic acids from a sample or fluid stream. In one embodiment, the sample or fluid stream comprises a waste or byproduct stream of an oil processing facility, such as OSPW. In one embodiment, the adsorbent may be used to treat mature fine tailings (MFT) or fluid fine tailings (FFT) to simultaneously consolidate the tailings while adsorbing contaminants in the liquid phase.

The keratin may be added in relatively small quantities to the waste water, MFT or FFT. In one embodiment, the keratin may be added in less than about 50 g/L of waste water, or 20 g/L, or 10 g/L. Alternatively, the keratin may be added in less than about 500 ppm, or 200 ppm, or 100 ppm of fluid or tailings volume.

As used herein, “trace metal” means metals present in a sample in small quantities, and may include one or more of Ni, Zn, Cu, Pb, Co, Se, V, Cr, or As. Naphthenic acids may comprise cycloaliphatic carboxylic acids, such as cyclopentyl and cyclohexyl carboxylic acids, typically with between about 10 to 16 carbon atoms.

In one embodiment, clean and finely divided keratin is modified by disrupting its tertiary structure, primarily by breaking the disulfide bonds. Some partial hydrolysis may occur, preferably while maintaining precipitation at a pH near its isoelectric point. In one embodiment, the modification comprises dissolution of the keratin in an alkaline or acidic solution, preferably with a reducing agent or an oxidizing agent. In one embodiment, the keratin is dissolved in NaOH, with Na2SO3 as a reducing agent.

Further modifications may be made, in order to introduce or expose functional groups which may enhance the absorption of contaminants such as trace metals and napthenic acids. Modifiers may react with or become complexed with the unfolded keratin molecules to provide additional functionality.

In one embodiment, the keratin may be further modified with a modifier intended to introduce more hydroxyl groups, for example, a polyol such as glycerol ethoxylate. In one embodiment, a modifier which disrupts disulfide bridges and bonds with the exposed thiol group may be used in an effort to prevent refolding of the keratin molecule. The thiol group may be presented with a cage structure such as a polyhedral oligomeric silsesquioxane (POSS) cage, preferably with hydrophobic substituents. Silsesquioxanes are hybrid inorganic-organic composite materials which combine physical properties of ceramics and functional group reactivity associated with organic chemistry. They may be produced by hydrolytic condensation of trifunctional silanes. In one embodiment, the POSS cage may comprise mercaptopropylisobutyl, which bears one thiol group and a plurality of hydrophobic organic substituents.

In one embodiment, the keratin may be further modified with iron based minerals, clays or layered silicates which bear multiple hydroxyl groups, such as smectites, vermiculite, kaolins, illite, chlorite, or iron oxyhydroxides such as hematite, goethite, lepidcrocite or ferrihydrite.

In one embodiment, the keratin may be further modified to enhance the number of carboxyl functional groups. Exemplary modifiers may include organic polyacids, such as tannic acid or citric acid.

In one embodiment, the keratin may be further modified with an aliphatic or aromatic diamine or polyamines. Preferably, at least one amine group is a primary amine, and the two amine groups are separated by at least two, three or four carbon molecules. In one embodiment, the diamine comprises 3-(dimethylamino)-1-propylamine.

### Examples

The following examples are only intended to illustrate specific embodiments of the claimed invention.

Materials and Methods

The analytical grade reagents sodium sulphite (Sigma-Aldrich, >98%, MW=125.04 g mol⁻¹), hydrochloric acid (Sigma-Aldrich, 37%), sodium hydroxide (Sigma-Aldrich, >97% pellets, MW=40 g mol⁻¹), mercaptopropylisobutyl POSS™, dioxane, tetrahydrofuran (THF), tannic acid, acetone, hexane, potassium hydroxide, glycerol ethoxylate and goethite were of commercial grade and used as received.

Feather Processing

White chicken feathers (CFs) were obtained from poultry research center at University of Alberta and they were washed several times with soapy hot water. The cleaned feathers were dried by spreading in a closed fume hood for one week to evaporate water and thereafter, they were placed in a ventilated oven for 24 h at 50° C. to completely remove remaining moisture. The hollow shaft, calamus were trimmed from vane of CFs using scissors.

Processed CFs were ground using a Fritsch cutting mill (Pulverisette 15, Laval Lab. Inc., Laval Canada) at a sieve insert size of 0.25 mm. The batches of ground CFs (20 g each) were further treated in Soxhlet (extraction tube with 50 mm internal diameter) for 5 h with 250 mL of Hexane. After evaporating hexane, dried CFs were stored in desiccator at room temperature.

Modification of Chicken Feathers

Chemical modification of CFs was carried out having in mind the cross-linked nature of keratinous protein due to disulfide linkages. Modifications were chosen to unfold protein, include molecules inside the biopolymer chain to provide additional functional groups, and/or to ensure that protein biopolymer does not fold back into its native cross-linked folded structure. In addition, chemical linkages of different functional groups can further enhance the surface functionality of keratin biopolymer.

Treatment with Alkaline Aqueous Solution (SM-01 or AM-204-A)

5 g purified CF was dissolved in 300 ml distilled water and 5 g reducing agent Na₂SO₃ and 20 ml 1M NaOH were added. The solution was stirred (150 RPM) at 150° C. temperature for few hours. After complete dissolution of keratin, 1M HCL was added to bring mixture close to isoelectric point of keratin (˜pH=4) and precipitation occurred. The mixture was centrifuged for 10 minutes and then the upper solution was removed. The sediment was washed with distilled water and centrifuged again. After multiple washing, the sediment was dried, ground and passed through 40 mesh sieves.

Modification of Keratin Biopolymer with Reducing Agent (MKBR)

In one liter round bottom flask, clean ground CF or keratin biopolymer (15 g) were mixed with 500 ml distilled water, followed by the addition of 24.87 g reducing agent Na₂SO₃, 90 g urea, 0.43 g EDTA, 12.1 g trisbase, and 4 mL of mercaptoethanol. The mixture was gently stirred at 70-80° C. temperature for one week. The solution was filtered to remove undissolved material and then dialyzed against water. The semi solid keratin biopolymer was dried at room temperature first and then in oven at 70° C. for twenty four hours. The dried keratin biopolymer was ground and passed through sieve prior to use for consolidation of tailings.

Treatment with Glycerol Ethoxylate (SM-02)

5 g purified CF was dissolved in 300 ml distilled water and 5 g Na₂SO₃ and 20 ml 1M NaOH were added. The solution was stirred (150 RPM) at 150° C. temperature for 2 hours. After 2 hours, sufficient 1M HCL was added to bring the pH to about 8. Then 125 mg of KOH and 3 ml of glycerol ethoxylate were added to the mixture and the mixture stirred (150 RPM) at 120° C. temperature for 3 hours. After this, 1M HCL was added to bring the mixture pH close to isoelectric point of keratin and precipitation occurred. The mixture was centrifuged for 10 minutes and then the liquid phase was removed. The sediment was washed multiple times and centrifuged. The sediment was dried, ground and passed through a sieve and stored in closed vial at room temperature until used.

Washing of CF (SM-03)

Finely ground neat (unmodified) CF was washed with hexane and dried completely to use as a reference.

Treatment with Mercaptopropylisobutyl POSS™ (SM-04)

3 g purified CF was dissolved in 300 ml distilled water and 5 g Na₂SO₃ and 20 ml 1M NaOH were added. The solution was stirred (150 RPM) at 150° C. temperature for 2 hours. Then 1M HCL was added to bring the solution to basic pH (˜pH 8). Then 0.3 g of Mercaptopropyllsobutyl POSS™ was added to the mixture and stirred (150 RPM) at 150° C. temperature for 2 hours. After 2 hours the temperature was reduced to 80° C. and 10 ml dioxane was added. The mixture stirred (150 RPM) at 150° C. temperature for further 2 hours. Then 1M HCL was added to bring the pH to ˜pH 4 and precipitation occurred. The mixture was centrifuged for 10 minutes and then the liquid phase was removed. The sediment was washed multiple times with THF to remove free POSS molecules and centrifuged. The upper solution was removed and the sediment was washed with distilled water multiple times and centrifuged. The sediment was dried, ground and sieved.

Modification of Keratin Biopolymer with POSS (MKBP)

In a 500 mL round bottom flask, ground chicken feathers (7 g) were mixed in 300 ml distilled water. Sodium sulphite (11.6 g) and urea (5 g) were also added into the reaction mixture. The reaction mixture was stirred at 75° C. for 3 hours, while the pH of the mixture was adjusted to slightly basic conditions by adding 1M solution of NaOH. Then mercaptopropylisobutyl-POSS (0.037 M) solution in 15 mL dioxane were added into the reaction mixture to be conjugated with the exposed functional groups in the keratin protein. For this purpose, the mixture was further stirred at 80° C. for 2 days. Then reaction contents were allowed to cool to room temperature and the pH of the solution was lowered to pH≈4 in order to precipitate the dissolved protein. The contents of the reaction mixture was centrifuged for 10 minutes and the supernatant was separated out. The sediment was thoroughly washed with THF to remove unbounded POSS molecules. Finally, the sediment was washed with distilled water multiple times and centrifuged. The sediment was dried in oven at 100° C., ground and sieved. The sieved MKBP biopolymer was further divided into two parts and were mixed in distill water. One part of it was treated with 1M solution of HCl to make it slightly acidic (MKBP-A), while the other part was treated with 1M solution of NaOH to make it slightly basic (MKBP-B). After getting the required pH, the supernatant of both fractions were removed after centrifugation, while the sediments were dried, ground and sieved before using them for consolidation of mature fine tailings.

Treatment with Goethite (SM-05)

5 g purified CF was dissolved in 200 ml distilled water in a 3-necked flask and 2 g Na₂SO₃ and 20 ml 1M NaOH were added. The solution was stirred (150 RPM) at 150° C. temperature for 2 hours. Then 1 g goethite was added and the temperature was reduced to 0° C. and purged with nitrogen. After 10 minutes 1 M HCL was added to bring the pH to 5. The flask was fitted with the condenser and was refluxed at 105° C. temperature for 24 hours. After 24 hours cold DI water was added to stop reaction and 1M HCL was added to bring the pH close to isoelectric point of keratin and the precipitation occurred. The solution was washed multiple times with 0.01 M HCL and then distilled water (DW). The mixture was centrifuged for 10 minutes and then the upper solution was removed and sediment was washed with water multiple times, dried, ground and sieved.

Treatment with Tannic Acid (SM-06)

5 g purified CF was dissolved in 200 ml distilled water and 2 g Na₂SO₃ and 20 ml 1M NaOH were added. The solution was stirred (150 RPM) at 150° C. temperature for 2 hours. Then the temperature was reduced to 0° C. and 1 g tannic acid was added. The mixture was stirred and then 1M HCL was added drop wise to bring the pH to 8. The flask was fitted with the condenser and refluxed at 150° C. temperature for 2 hours. Then cold DI water was added to stop reaction and 1M HCL was added to bring the PH to 4 and the precipitation occurred. The mixture was centrifuged for 10 minutes and then the upper solution was removed. The sediment was washed several times with acetone and then DW. Then the sediment was dried, ground, sieved and stored in sealed vial till used.

Treatment with 3-(Dimethylamino)-1-Propylamine (AM-205 or MKBD)

For modification of keratin biopolymer with 3-(dimethylamino)-1-propylamine, 5 g purified ground chicken feathers were mixed in 150 ml methanol, followed by the addition of 5 g Na₂SO₃, 20 g urea, 100 mg EDTA, 4 g trisbase, and mercaptoethanol (1.8 mL). The mixture was stirred at 65° C. for 24 hours. Then 3-(dimethylamino)-1-propylamine was added and the reaction mixture was further heated to reflux for additional 24 hours. After that, the mixture was filtered and thoroughly washed with methanol and water respectively. The material was dried, ground and sieved before using for mature fine tailings consolidation.

Co-Polymerization of Canola Fatty Acid with NiPAM (AM-203)

Canola fatty acids derived monomer (50%) and N-isopropylacrylamide (NiPAM) (50%) were taken in a round bottom flask and purged with nitrogen gas for ten minutes to create an inert environment before adding an initiator azobisisobutyronitrile (AIBN) (1%). The reaction mixture was kept in a preheated oil bath having 70° C. temperature and stirred for 16 hours to ensure complete polymerization. Copolymer was washed multiple times with THF to remove the unreacted contents and smaller molecules of polymer. Finally, the copolymer was dried, ground and used for consolidation analysis.

Morphological Changes—SEM Characterization

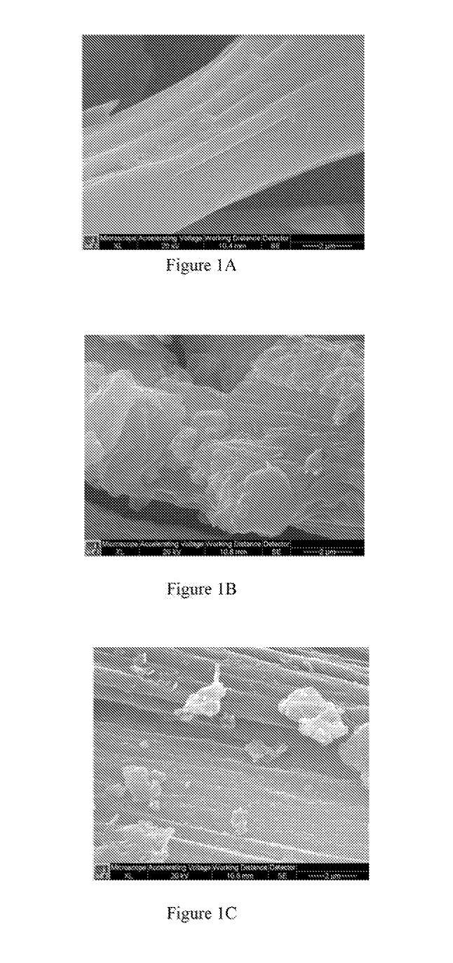

Scanning electron microscopy (SEM) images were scanned with a Philips-FE! model Quanta 20 to study morphological changes of the feather keratin and chemically modified feather keratin-based sorbents. As illustrated in FIG. 1A, unmodified feather remains intact with smooth surface. While in-situ modified feather keratins (FIGS. 1B and 1C) show increased surface roughness with micro-cracks and the shiny patches on the surface. On modification, fiber changed its structure, and causes roughness that is the characteristics of conformational change and unfolding of cross-linked structure as compared to unmodified keratin fiber. Interestingly, modifications mostly resulted on complete loss of structural integrity of feather keratin leading to more amorphous structure with increased roughness or in some cases, depending upon modifier, only a small proportion of feather still retained their structure after treatment. These modified keratin forms were characterized and marked with higher sorption properties and affinity for certain contaminants.

X-RD Characterization

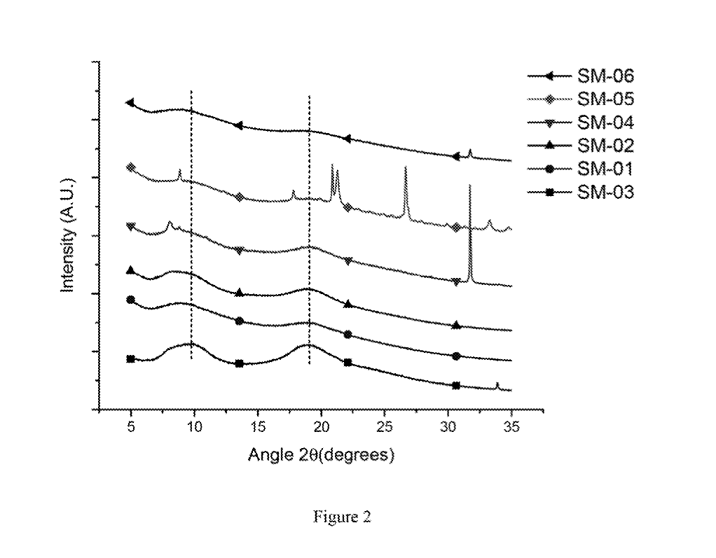

X-ray powder diffraction pattern were recorded using Rigaku Ultima IV, Geigerflex Powder Diffractometer with Cu-Kα radiation (λ=0.154 nm) to investigate the crystallinity. Wide angle X-ray diffraction (WAXD) patterns of untreated keratin biopolymer and modified materials were studied. As shown in FIG. 2, the native keratin SM-03 displayed a typical keratins pattern with a prominent 20 peak at 9.9° that corresponds to the a-helix configuration and more intense band at 19° indexed as its beta strand secondary structure. Surface modifications show substantial reduction in both a-helix and beta strand peaks, indicating some major changes in crystalline structures. In such cases, the reductions and/or disappearance in both a-helix and beta strands corresponds to fracture of a-helix and beta-sheet crystalline network suggesting that crystalline structures were mostly destroyed by modification leading to amorphous structures. This strengthens the idea of higher the decrease in crystallinity would lead to higher modification of keratin material.

Some new crystallinity peaks appeared in SM-04 and SM-05S suggesting new rearrangements of polymer chains leading to new crystalline regions.

FTIR Characterization

Fourier transformed infrared (FTIR) spectra of chicken feather and modified sorbents were obtained on a FTIR (Bruker Vertex 70, Billerica, Mass., USA) with an attached Hyperion 2000 FTIR Microscope spectrometer fitted with a germanium attenuated total reflection (ATR) microscope objective. A mercury cadmium telluride (MCT) detector was used. The spectra were collected within the frequency range 4000-500 cm⁻¹, under the same conditions as the background. All sample spectra were recorded at 128 scans and 4 cm⁻¹ resolution, and spectra of two replicate measurements for each sample were averaged. The infrared spectra were acquired using Bruker OPUS software (version 5.5) and analyzed by using Thermo Scientific OMNIC software package (version 7.1).

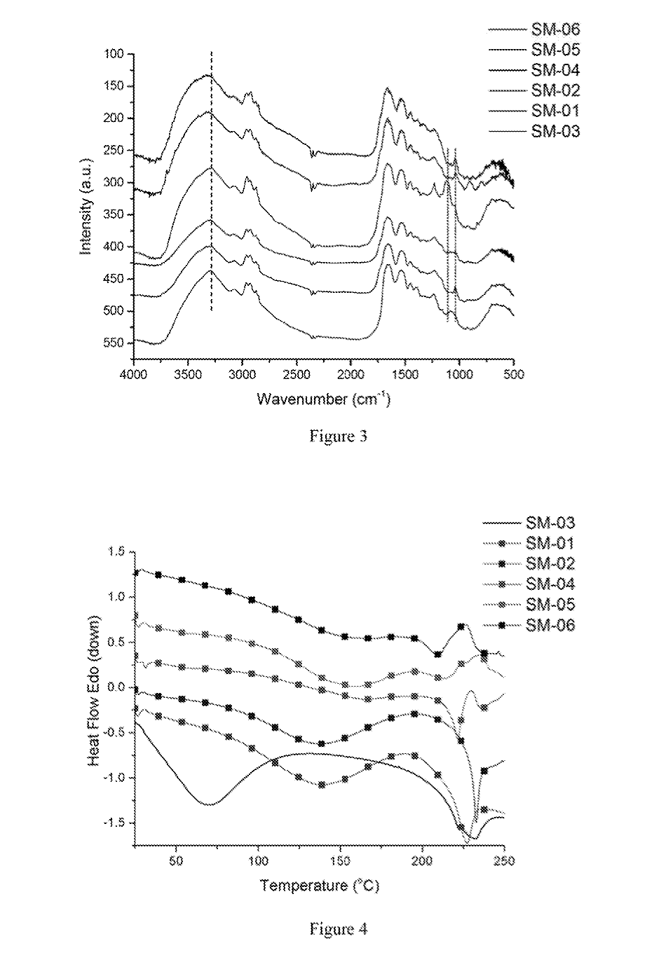

FTIR investigation can be used as an effective tool to assess the structural changes in proteins. In FIG. 3, the IR spectra of neat keratin biopolymer and modified sorbents exhibit typical amide vibrations including amide A (N—H stretching, 3300 cm⁻¹), amide I (C═O stretching, with a minor contribution from N—H bending and C—N stretching, 1600-1700 cm⁻¹), amide II and amide III (N—H bending and C—N stretching, at around 1540 and 1240 cm⁻¹, respectively). In IR spectra of all modified samples especially SM-01, 04, 05, and 06, appearance of new peak at 1031 cm⁻¹ approx. is attributed to vibrational stretching of polar and unsaturated residues such as C—O, C═C, and CC—O of polypeptide side chains. Similarly a peak centered at around 3295 cm⁻¹ in neat keratin shifts to higher wave number and becomes broad in modified materials suggesting changes in arrangement and nature of H-bonding in native keratin. In addition, appearance of shoulder at around 1734 cm⁻¹ in some modified samples, indicates esterification of some of the carboxyl groups of keratin.

Thermal Properties—Differential Scanning Calorimetry (DSC) and Thermogravimetric Analysis (TGA) Characterizations

The thermograms were performed under a continuous nitrogen purge on a (Modulated 2920, TA Instrument, USA) calorimetric apparatus. Pure indium sample was used to calibrate the heat flow and temperature of the instrument. All samples were scanned in a temperature range of 25-270° C. at a rate of 5° C. per minute. Samples having a mass of ˜5 mg were scanned at 10° C./min from 0 to 250° C. TOA was performed on a Q50 (TA Instrument, USA) thermogravimetric analyzer. About 10 mg of the sample was heated at 10° C./min over a temperature range of 25-600° C. under a nitrogen atmosphere. The thermal transitions of the whole feather as well as surface and solution modified materials were studied by DSC.

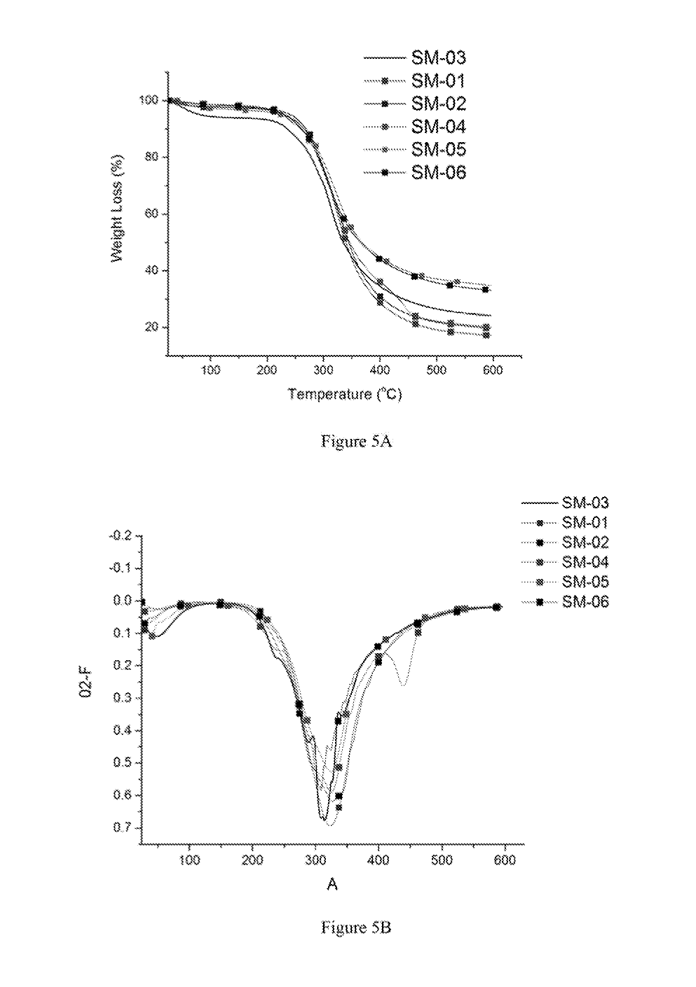

Typical heat flow curves of feather and their corresponding modified materials are shown in FIG. 4. The DSC trace for raw CF has higher heat flow values as compared to modified materials. A low temperature broad peak at around 100° C. is indicative for denaturation/evaporation of residual moisture of the protein. This curve for modified materials shows distinctively different behavior with wider range of denaturation from its neat keratin suggesting modification and change in exposure of functional groups. The DSC of untreated CF shows an exothermic peak around −230° C., which is usually assigned to a-helix disordering and decomposition. However, the modified chicken feather materials show relatively sharper peaks shifted to comparatively lower temperatures.

These observations suggest the loss of ordered helix and sheet structures and gain of relatively amorphous behavior in all modified material especially marked with lower peak intensity and broaden melt curve trend.

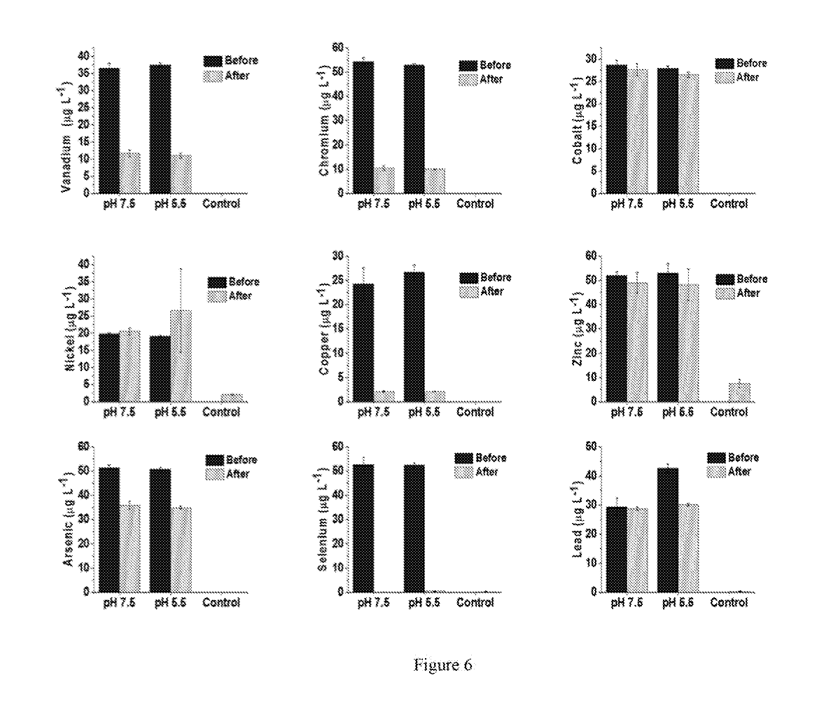

The TG curves of neat feather and their modified materials are shown in FIG. 5. Two weight loss steps can be seen in case of keratin materials. The weight loss in the first stage (near 100° C.) was due to the evaporation of residual water whereas the second step (between 250 and 600° C.) was mainly due to the degradation of the keratin.

The degradation of modified materials consisted of two and in some cases three weight loss steps. The first gradual weight loss (below 150° C.) is due to the evaporation of moisture, the second (between 150 to 250° C.) is attributed to the modifier evaporation which was not chemically linked to keratin, and the final weight loss beyond 250° C. is due to decomposition of keratin material. A clear increase in thermal stability was observed in successful modified materials.

Metal Sorption—Synthetic Waste Water Experiment:

A high ionic strength (0.05) solution representative of industrial wastewater was designed (synthetic waste water). This solution consisted of sodium chloride (1400 mg L⁻¹), calcium chloride (1000 mg L⁻¹), and nine different trace elements (Ni, Zn, Cu, Pb, Co, Se, V, Cr, As) added at 50 μg L⁻¹ each. In the case of certain redox sensitive elements (As, Se, Cr, V), the more toxic chemical oxidation state of the element was added. These included As(III), Se(IV), Cr(VI) and V(V). The synthetic waste water was split in two 500 ml aliquots with the pH adjusted using 0.1 N NaOH drop wise in one aliquote to pH 5.5 and pH 7.5 in the other aliquot.

0.1 grams of sorbents SM-01, SM-03 and SM-06 were weighed in triplicate and filled with 10 mL of appropriate synthetic water. To see the potential contributions of these elements from the sorbent itself, a control was added (in triplicate) where 10 mL of Millipore water (resistivity 18.2 MΩ) was added to 0.1 grams of sorbent. Samples were placed on a reciprocating shaker and gently agitated for 24 hours. Samples were then centrifuged and the supernatant was analyzed using inductively coupled plasma mass spectrometry (ICP-MS; iCap Q Thermo Scientific) with appropriate internal and external standards. Additional quality control measures included labware blanks, water blanks and SLRS-5 reference material.

Initial experiments did not yield reliable quantitative data, but did allow optimization of method protocol and the selection of appropriate storage containers and methods for analyzing on ICP-MS. The greatest issues came from unreliable initial concentrations of trace elements. Borosilicate glass containers were determined to be a sink for several elements which were adsorbed to the container over time. Even greater variability with the samples that contained naphthenic acids, likely due to having these samples in polypropylene centrifuge tubes.

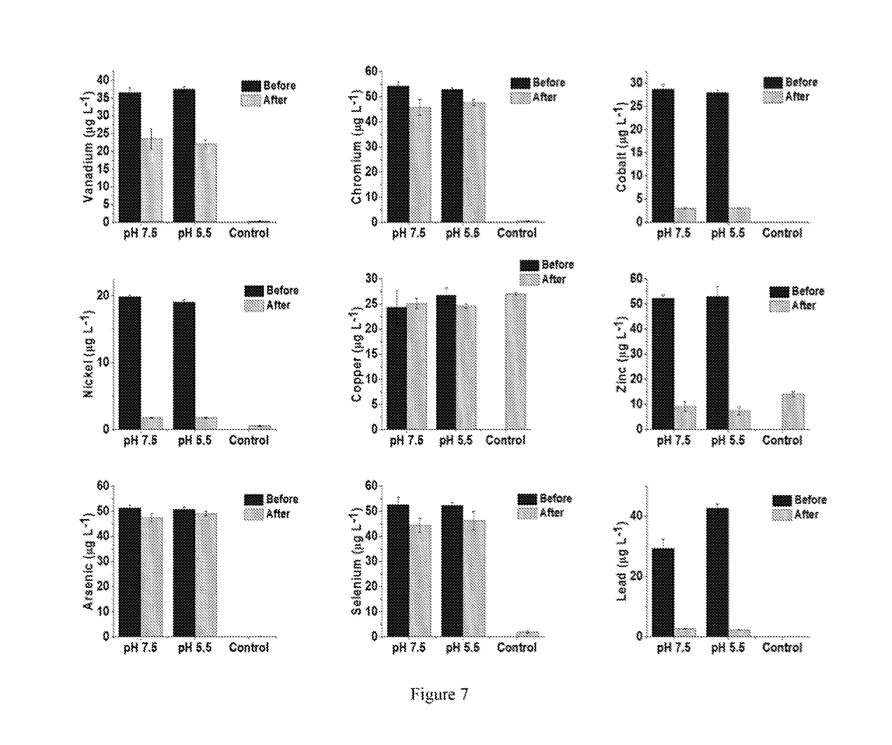

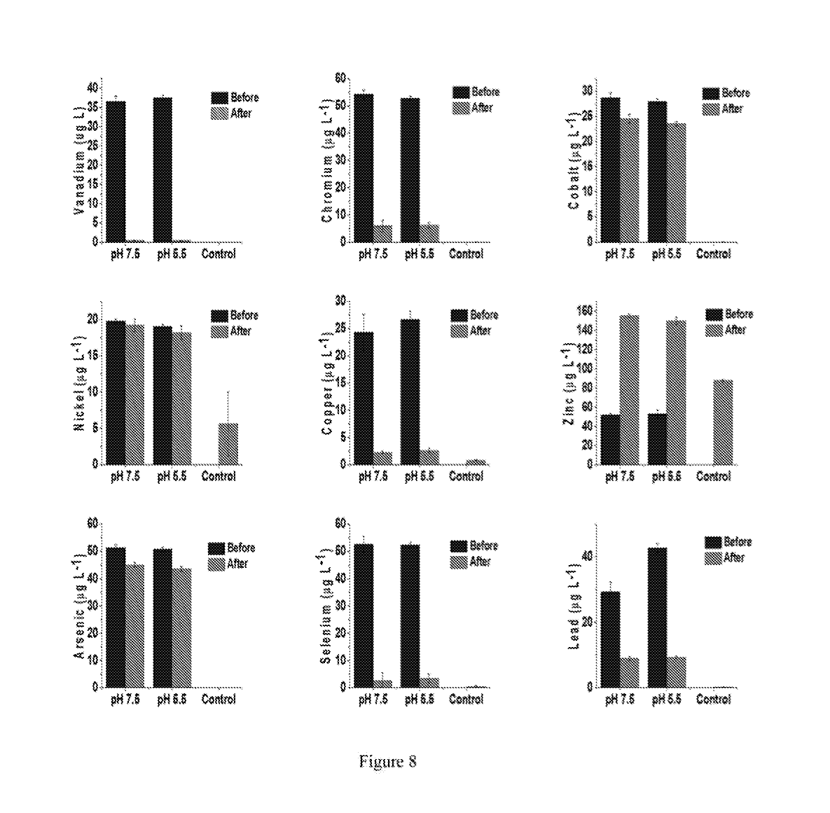

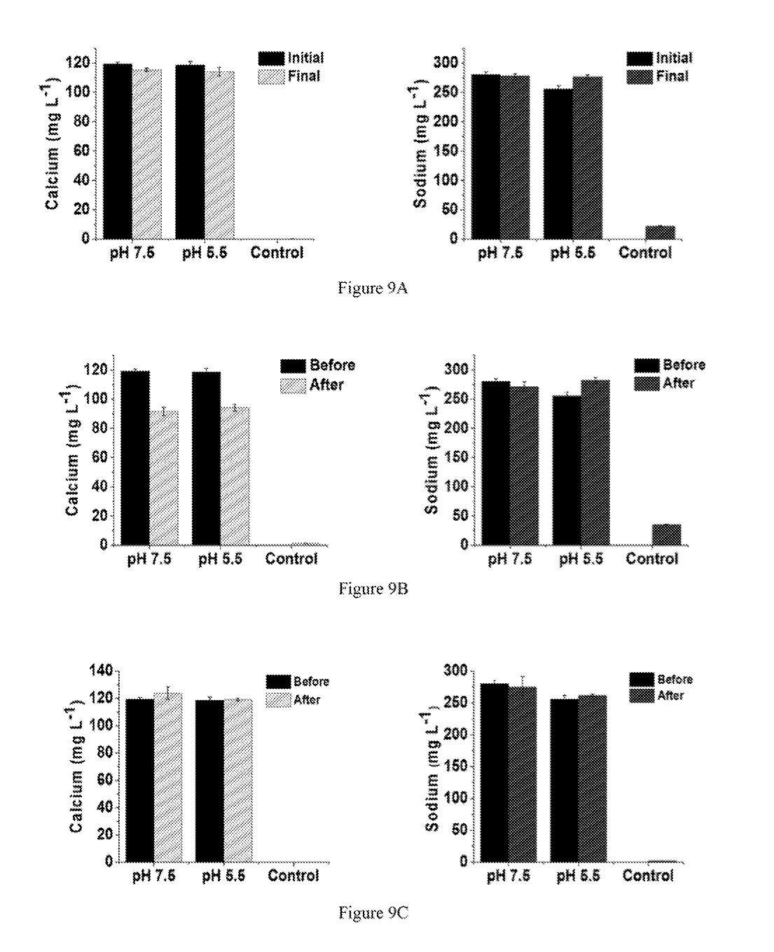

The results demonstrate the affinity of certain sorbents for trace elements depending on the chemical modification. It is clear (FIGS. 6-8) that the chemical modification plays a direct role in the removal of certain elements in solution.

Sorbent material #1 (SM-01) displayed a strong affinity for V, Cr, Cu, and Se

Sorbent material #3 (SM-03) displayed a strong affinity for Co, Ni, Zn, and Pb

Sorbent material #6 (SM-06) displayed a strong affinity for V, Cr, Cu, Se and Pb

In general, the sorbent materials SM-01 and SM-06 displayed greater affinity towards elements present as anions in solution. While the positively charged cations showed more attraction towards the sorbent (SM-03).

Another important aspect to consider is the selectivity of the sorbents. Despite being in a solution that is concentrated with Ca, Na and Cl ions, the sorbents were still successful in removing the desired trace elements. This may be a very useful property as industrial waste waters often contain an abundance of common salts that are non-toxic and pose little threat to the environment. Many trace elements (or trace metals as they are commonly referred to) are present in low amounts compared to more common ions such as Ca, Na and Cl and can be difficult to selectively remove. Greater amounts of sorbent material may be needed to capture these trace elements to remove them so they are present in a safe concentration.

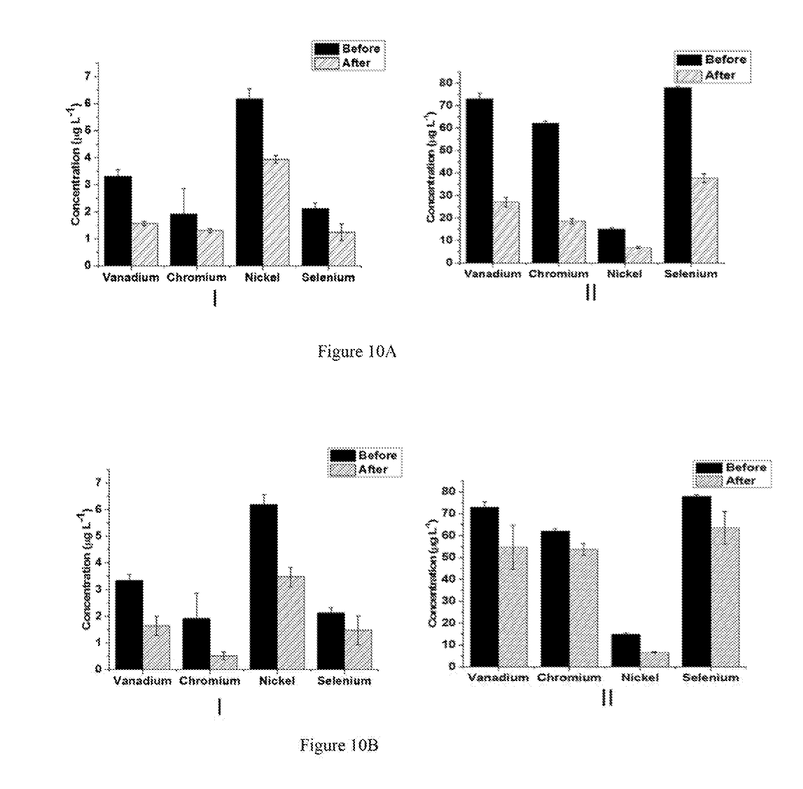

We observed relatively stable concentrations of Ca and Na and showed no signs of being strongly adsorbed to the sorbents (FIGS. 9A, 9B, 9C). Only SM-03 displayed adsorption for Ca (FIG. 9A), which is likely due to the apparent attraction for cationic compounds from the surface groups. While the concentration of Na did not decrease in the presence of SM-03, there may be some adsorption of Na which is off-set an amount of Na leaching from the sorbent in the control samples.

Oil Sands Process Water (OSPW) Experiment

A. Sorption of Metals:

An experimental design similar to synthetic waste water metal sorption was used to evaluate the effectiveness of the biosorbent with oil sands process affected water. The process water (stored at 4 degrees C.) was brought to room temperature then divided into two 500 ml volumes. One portion was spiked with Ni, Cr(VI), Se(VI), and V(V). The other was left without the addition of these elements to remain as a control. The same volume, sorbent types, and sorbent amounts were used here as in the synthetic water experiment. Only these elements were chosen to be analyzed to first assess the effectiveness and adapt/optimize our method for using OSPW as a sample matrix.

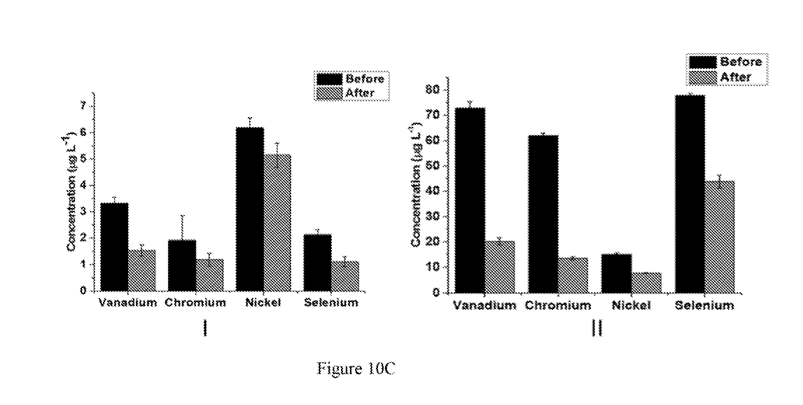

The results of this experiment were similar to the synthetic waste water experiment in terms of the different sorbent affinity for certain trace elements. OSPW is a complex mixture of inorganic and organic compounds that interact in many different ways. However, OSPW differs from the synthetic waste water as it includes a high concentration of organic compounds such as naphthenic acids. There are also high amounts of other elements including (but certainly not limited to) Sr, Al, Fe, Zn, Ga, Ba and Rb. All these components of OSPW may be competitive for adsorption sites that might be meant for specific elements of concern. In preliminary results (FIGS. 10A, 10B, 10C) we found that in high concentrations, the sorbents removed a large amount of the targetted element. SM-01 (10A), SM-03 (10B) and SM-06 (10C). Graph I displays the treatment of oil sands process-affected water without any addition of metals V, Cr, Ni or Se. Graph II displays the sorption of these elements when the sample is spiked with high concentration of metals.

B. Sorption of Naphthenic Acids (NAs):

0.1 g of each sorbent (SM-01 to 06) was weighed separately in a glass vial followed by the addition of 10 mL of OSPW. Samples were placed on a reciprocating shaker and gently agitated for 24 hours. The blank samples of OSPW (without bio-sorbent), were shaken in the same vials to offset the sorption of blank vial if any and to be used as a reference. After shaking, all these samples were centrifuged for 10 minutes and supernatant was collected.

Extraction and Estimation of NAs.

The pH of the collected samples was adjusted to ˜pH 10 by adding 1M solution of NaOH. At basic pH, the acids remain deprotonated to their conjugate bases and are completely soluble in the water. Then dichloromethane (10 mL×2) was added into the aqueous layer to extract the components other than acids. After that, the aqueous layer was acidified with 1M solution of HCl to adjust the pH approximately equal to ˜2, which results in the protonation of acids to make them soluble in organic solvents. Naphthenic acids were extracted from this acidified aqueous layer by using dichloromethane (10 mL×2). The combined organic layers were dried with anhydrous sodium sulphate.

The concentration of NAs obtained from OSPW after treatment with sorbents was measured by FTIR spectroscopy. Dichloromethane was evaporated by keeping the sample vials in the fuming hood overnight. Then the measured quantity of dichloromethane (3 mL) was added in each sample to dissolve the extracted naphthenic acids. The sample vials were capped tightly before proceeding to FTIR analysis.

The absorbance at 1743 and 1706 cm⁻¹ due to monomeric and dimeric forms of the carboxylic groups measured respectively. The sum of these two absorbances was compared to calibration curve prepared by known concentration of Merichem naphthenic acid standards. Due to variation of naphthenic acid concentration in different OSPW samples, six measurements of OSPW were carried out and average value of six measurements has been reported. The calibration standards were prepared by dissolving stock solution of Merichem naphthenic acids in dichloromethane (DCM) to get a concentration range from 50 to 500 mg/L in a final volume of 10 mL DCM. The standards were analyzed in the same way as mentioned above.

The results in the FTIR spectra show that the sample with high concentration of naphthenic acid had higher absorbance and their peak intensity is high, while those with low concentration showed less absorbance band with low peak intensity. From the spectrum results, it can be observed that the peak intensity of sorbent sample SM-04 is lowest compared to all others, which represents its maximum tendency to adsorb naphthenic acids. While, SM-06 showed high absorbance value in the FTIR spectra, which represents the high concentration of naphthenic acids. It is apparent form the results that SM-04 and SM-05 had better sorption of NAs, compared to the concentration of reference sample of OSPW. Both of these samples were much more effective in sorption of NAs compared to SM-01, SM-02 and SM-03. To further understand the sorption efficiency, the OSPW was diluted to different concentrations and two sorbents SM-04 (POSS modified keratin biopolymer (PMKB)) and SM-05 (Goethite modified keratin biopolymer (GMKB)) were tested. The POSS and goethite supported chemical modifications of keratin fiber have potential to remove NAs from OSPW and their respective highest biosorption capacities of 4450 and 4880 were obtained at maximum concentration of NAs in OSPW. Conversely, modified PMKB and GMKB biosorbents gave maximum 64.6% and 66.1% rejection percentages respectively in response to lower concentrations of NAs in OSPW for the fixed amount of 0.1 g dose of tested biosorbents (Arshad et al. 2016)

Flocculation/Settling Consolidation

For the consolidation of mature fine tailings (MFT), a required dosage of biopolymers were mixed in 200 ml, volume of MFT and stirred for few minutes at room temperature. These mixtures of MFT containing biopolymer were transferred into 500 mL graduated cylinder. For each test, MFT was also treated with synthetic polyacrylamide (PAM) for comparison. A negative (blank) column of MFT was also monitored to compare settlings without any flocculent. The settling/consolidation of fine particles and water release were measured after regular intervals for several days. Two different experiments with different biopolymers for settlings of mature fine tailings were performed. In first experiment, biopolymers MKBP-A and MKBP-B were used with 500 ppm concentration for a volume of 200 mL MFT. In second experiment, MKBR, MKBD and CFA-NiPAM were tested with 200 ppm concentration against 200 mL volume of MFT.

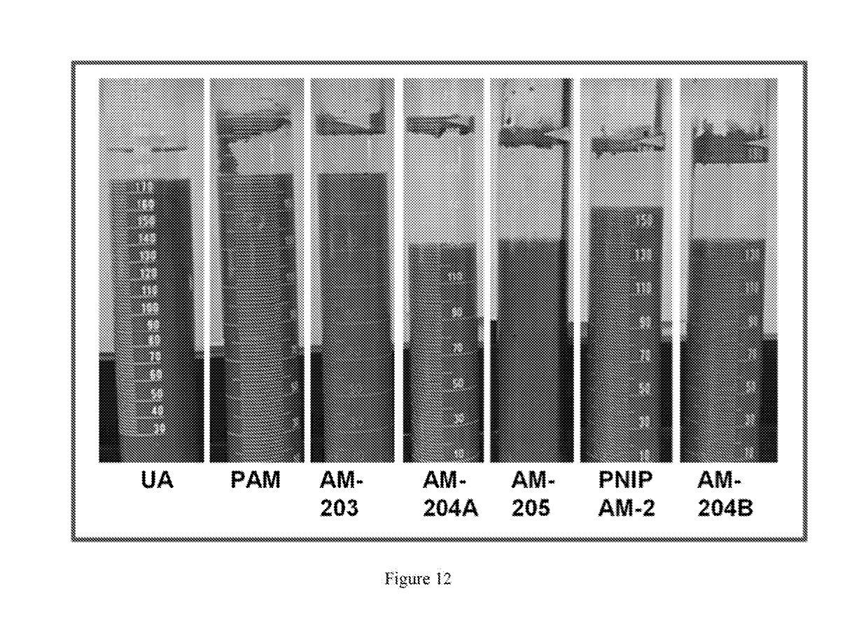

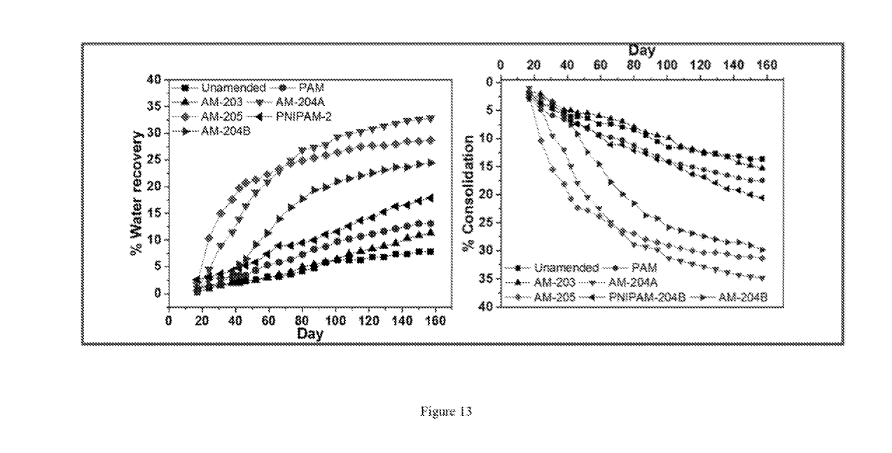

Five biopolymers (AM-203, AM-204A, AM-205, PNIPAM-2 and AM-204B) with different modifications to fine tune surface functionality were tested by amending fluid fine tailings (FFT) with biopolymers at 200 mg kg⁻¹ and compared with an unamended FFT (UA) and FFT amended with synthetic polymer (PAM) for flocculation of 200 mL of FFT that initially contained 25% solids (FIG. 1). The experiment was conducted for ˜157 days to assess performance of biopolymers. Most biopolymers yielded very promising results by consolidating FFT (21-35%) as may be seen in FIG. 12, and recovering large volumes of porewater (18-33%) greater than unamended FFT (13% and 8%, respectively) and PAM amended FFT (17% and 13%, respectively) (FIG. 13).

Porewater recovered from biopolymer amended FFT was clear with negligible suspended particles (FIG. 12). The results show that the biopolymers developed for the consolidation of tailings may have exceptional capabilities with multiple advantages including better consolidation, higher pore water recovery along with removal of metals and naphthenic acids.

### REFERENCES

The following references are incorporated herein by reference, where permitted, as if reproduced in their entirety.
1. http://www.energy.gov.ab.ca/OilSands/oilsands.asp.
2. Scott, A. C., R. F. Young, and P. M. Fedorak, Comparison of GC-MS and FTIR methods for quantifying naphthenic acids in water samples. Chemosphere, 2008. 73(8): p. 1258-1264.
3. http://www.oilsands.alberta.calwater.html.
4. Allen, E. W., Process Water Treatment in Canada's Oil Sands Industry: I Target Pollutants and Treatment Objectives. Journal of Environmental Engineering and Science, 2008. 7: p. 499-524.
5. Kasperski, K. L., Review of research on aqueous extraction of bitumen from mined oil sands. Unpublished report., 2003. CANMET Energy Technology Centre, Natural Resources Canada, Devon, Alta.
6. MacKinnon, M. D. and H. Boerger, DESCRIPTION OF TWO TREATMENT METHODS FOR DETOXIFYING OIL SANDS TAILINGS POND WATER. Water pollution research journal of Canada, 1986. 21(4): p. 496-512.
7. Allen, E. W., Process water treatment in Canada's oil sands industry: I Target pollutants and treatment objectives. Journal of Environmental Engineering and Science, 2008. 7(2): p. 123-138.
8. Hansen BR, D. S., Review of potential technologies for the removal of dissolved components from produced water. Chern. Eng. Res. Des., 1994.72: p. 176-188.
9. Gallup, D. L., E. G. Isacoff, and D. N. Smith, Use of Ambersorb® carbonaceous adsorbent for removal of BTEJX compounds from oil-field produced water. Environmental Progress, 1996. 15(3): p. 197-203.
10. Guibal, B., Interactions of metal ions with chilosan-based sorbents: a review. Separation and Purification Technology, 2004. 38(1): p. 43-74.
11. Hartley, F., Studies in chrome mordanting. II The binding of chromium(III) cations to wool. Australian Journal of Chemistry, 1968. 21(11): p. 2723-2735.
12. McGovern, V., Recycling Poultry Feathers: More Bang for the Cluck. Environmental Health Perspectives, 2000. 108(8): p. A366-A369.
13. Gupta, V. K., et al., Adsorption of a hazardous dye, erythrosine, over hen feathers. Journal of Colloid and Interface Science, 2006. 304(1): p. 52-57.
14. Mittal, A., Adsorption kinetics of removal of a toxic dye, Malachite Green, from wastewater by using hen feathers. Journal of Hazardous Materials, 2006. 133(1-3): p. 196-202.
15. Al-Asheh, S., F. Banat, and D. Al-Rousan, Adsorption of Copper, Zinc and Nickel ions from Single and Binary Metal Ion Mixtures on to Chicken Feathers. Adsorption Science & Technology, 2002. 20(9): p. 849-864.
16. Banat, F., S. Al-Asheh, and D. Al-Rousan, Comparison between Different Keratin-composed Biosorbents for the Removal of Heavy Metal Ions from Aqueous Solutions. Adsorption Science & Technology, 2002. 20(4): p. 393-416.
17. Khosa, M. A. and A. Ullah, In-situ modification, regeneration, and application of keratin biopolymer for arsenic removal. Journal of Hazardous Materials, 2014. 278(0): p. 360-371.
18. Khosa, M. A., J. Wu, and A. Ullah, Chemical modification, characterization, and application of chicken feathers as novel biosorbents. RSC Advances, 2013. 3(43): p. 20800-20810.
19. Kar, P. and M. Misra, Use of keratin fiber for separation of heavy metals from water. Journal of Chemical Technology & Biotechnology, 2004. 79(11): p. 1313-1319.
20. Ullah, A., et al., Bioplastics from Feather Quill. Biomacromolecules, 2011. 12(10): p. 3826-3832.
21. Crini, G. and P.-M. Badot, Application of chitosan, a natural aminopolysaccharide, for dye removal from aqueous solutions by adsorption processes using batch studies: A review of recent literature. Progress in Polymer Science, 2008. 33(4): p. 399-447.
22. T. Y. Ding, S. L. H., L. G. A. Ong, Comparison of pretreatment strategies for conversion of coconut husk fiber to fermentable sugars. Bioresourees, 2012. 7(2): p. 1540-1547.
23. M. Arshad, M A Khosa, T. Siddique, A. Ullah, Modified biopolymers as sorbents for the removal of naphthenic acids from oil sands process affected water (OSPW). Chemosphere 163 (2016)

What is claimed is:

1. A method of removing trace metals and/or napthenic acids from a waste water stream, comprising the step of contacting the waste water stream with keratin.

2. The method of claim 1 wherein the waste water stream comprise mature or fine fluid tailings from oil sands operations, wherein the method comprises the removal of trace metals, naphthenic acids with simultaneous consolidation of the tailings.

3. The method of claim 2 wherein the keratin is modified by a denaturing step.

4. The method of claim 3 wherein the keratin is denatured and partially hydrolyzed in an alkaline or acid solution, optionally with a reducing agent or an oxidizing agent, and precipitated at about its isoelectric point.

5. The method of claim 3 wherein the keratin is further modified by a polyol.

6. The method of claim 5 wherein the polyol comprises glycerol ethoxylate.

7. The method of claim 3 wherein the keratin is further modified by a modifier bearing a a thiol group.

8. The method of claim 7 wherein the modifier comprises a 6 wherein the thiol group is borne on a cage structure.

9. The method of claim 8 wherein the cage structure comprises a silsesquioxane.

10. The method of claim 9 wherein the silsesquioxane comprises a polyhedral oligomeric silsesquloxane cage with organic hydrophobic substituents and one or more of a hydroxyl, amine or a carboxyl substituent.

11. The method of claim 3 wherein the keratin is further modified by a mineral, clay or silicate which bears multiple hydroxyl groups.

12. The method of claim 11 wherein the mineral, clay or silicate comprises a smectite, vermiculite, kaolin, illite, chlorite, or iron oxyhydroxide.

13. The method of claim 12 wherein the iron oxyhydroxide comprises hematite, goethite, lepidcrocite or ferrihydrite.

14. The method of claim 3 wherein the keratin is further modified by or an organic polyacid.

15. The method of claim 14 wherein the organic polyacid comprises tannic acid or citric acid.

16. The method of claim 3 wherein the keratin is further modified by a diamine or polyamine, wherein at least one amine group is a primary amine.

17. The method of claim 16 wherein the diamine is 3-(dimethylamino)-1-propylamine.

18. A modified keratin adapted for use as an absorbent to remove trace metals and/or napthenic acids from a waste water stream.

19. The modified keratin of claim 18 adapted for use as an adsorbent to remove trace metals and napthenic acids, while simultaneously a flocculent to consolidate oil sands tailings.

20. Use of a modified keratin to (a) remove trace metals and napthenic acids from a waste water stream, or (b) consolidate oil sands tailings, or (c) both simultaneously.
